(19) United States
(12) Patent Application Publication
(10) Pub. No.: US 2013/0197386 A1
(43) Pub. Date: Aug. 1, 2013

(54) RESPIRATORY FUNCTION DETECTION

(71) Applicant: Medtronic, Inc., Minneapolis, MN (US)

(72) Inventors: Yong K. Cho, Maple Grove, MN (US); Mark K. Erickson, Brooklyn Park, MN (US); Douglas A. Hettrick, Andover, MN (US)

(73) Assignee: MEDTRONIC, INC., Minneapolis, MN (US)

(21) Appl. No.: 13/744,461

(22) Filed: Jan. 18, 2013

Related U.S. Application Data
(60) Provisional application No. 61/593,042, filed on Jan. 31, 2012.

(51) Int. Cl. A61B 5/085; A61B 5/08

(52) U.S. Cl. CPC A61B 5/085; A61B 5/08
USPC 600/533; 600/529

(57) ABSTRACT

A system and method for monitoring respiratory function that includes an acoustic sensing device sensing an acoustic waveform ocurring during one of an inspiration phase associated with at least one breath of a patient and an expiration phase associated with at least one breath of a patient, and a processor configured to determine changes in high frequency acoustic amplitude associated with the sensed acoustic waveform and, in response to the determined changes in high frequency acoustic amplitude, determine an indication of respiratory function.

### RELATED APPLICATION

The present application claims priority and other benefits from U.S. Provisional Patent Application Ser. No. 61/593,042, filed Jan. 31, 2012, entitled “RESPIRATORY FUNCTION DETECTION” (Attorney Docket P0029560.USP1), incorporated herein by reference in its entirety.

### CROSS-REFERENCE TO RELATED APPLICATIONS

Cross-reference is hereby made to the commonly-assigned related U.S. application Ser. No. ______ (Attorney Docket Number P0029560.USU2), entitled “RESPIRATORY FUNCTION DETECTION,” to Cho et al., filed concurrently herewith and incorporated herein by reference in it's entirety.

### TECHNICAL FIELD

The disclosure relates to implantable medical devices, and more particularly implantable medical devices that monitor respiration.

### BACKGROUND

Apnea is a relatively common form of disordered breathing characterized by interruptions to a patient's breathing. Often, the interruptions to breathing occur during sleep, at which time the disorder is called sleep apnea. Breathing cessation may occur numerous times during sleep, in some cases hundreds of times a night. Each cessation of breathing may last for up to a minute or longer.

Sleep apnea has multiple classifications based on the source of the dysfunction. For example, central sleep apnea (CSA) results from neurological dysfunction, while obstructive sleep apnea (OSA) results form a mechanical blockage of the airway. The mechanical blockage may be due, for example, to fatty neck tissue compressing the trachea. In addition to sleep apnea, there are other types of disordered breathing, including hypopnea (shallow breathing), hypernea (heavy breathing), tachypnea (rapid breathing), dyspnea (labored breathing) and orthopnea (difficulty breathing when lying down).

Cheyne-Stokes respiration is a specific form of abnormal breathing resulting in apneic intervals. Cheyne-Stokes respiration is characterized by progressively deeper and sometimes faster breathing that is followed by a gradual decrease in tidal volume, resulting in a temporary stoppage in breathing. Hence, Cheyne-Stokes respiration has been described as an oscillation of ventilation between apnea and hypernea.

There is a high comorbidity between these various types of disordered breathing and congestive heart failure. Pulmonary edema is an abnormal buildup of fluid within the lung that may lead to shortness of breath. Pulmonary edema may be caused by congestive heart failure. As the heart grows weaker, pressure in the veins leading to the lungs start to rise. As pressure in the blood vessels rises, fluid is pushed into the air spaces in the lungs. The fluid may interrupt normal oxygen movement throughout the lungs, leading to changes in breathing patterns and a feeling of shortness of breath.

### BRIEF DESCRIPTION OF DRAWINGS

The details of one or more examples in this disclosure are set forth in the accompanying drawings and the description below. Other features, objects, and advantages of the disclosure will be apparent from the description and drawings, and from the claims.

FIG. 1 is a conceptual diagram illustrating an example system that measures physiological events associated with hemodynamic or respiratory function.

FIGS. 2A and 2B are conceptual diagrams illustrating example IMDs.

FIG. 3 is a conceptual diagram illustrating another example system that measures physiological events associated with hemodynamic or respiratory function.

FIG. 4 is a block diagram illustrating an example configuration of an IMD, such as the example IMDs illustrated in FIGS. 1-3.

FIG. 5 is a block diagram illustrating an example configuration of a signal analyzer of the IMD of FIG. 4.

FIG. 6 is a block diagram illustrating an example system that includes an external device such as a server, and one or more computing devices that are coupled to an IMD and programmer, such as the IMDs and programmers shown in FIGS. 1-4.

FIG. 7 is a flow chart illustrating an example mode of operation of an IMD for assessing respiratory function using impedance signals

FIG. 8 is a flow chart illustrating another example mode of operation of an IMD for assessing respiratory function using impedance signals.

FIGS. 9A through 9D is an example progression of impedance and acoustic waveforms through the diagnosis and treatment of pulmonary edema.

FIG. 10 is a graph of experimental data illustrating changes in real-time impedance and intratracheal pressure.

FIG. 11 is a linear regression plot of subcutaneous impedance compared to intratracheal pressure.

FIG. 12 is a graph illustrating a conductance waveform.

### DETAILED DESCRIPTION

In general, the disclosure is directed to techniques for detecting and monitoring the respiratory function of a patient, e.g., based on detection of abnormal breathing patterns. In some examples, abnormal breathing patterns or other changes in patient status are detected based on impedance signals. In some examples, thoracic auscultations may also be used in detecting abnormal breathing patterns or changes in patient status.

In one example, the disclosure is directed to a method including receiving a first thoracic impedance waveform for at least one breath of a patient; determining a first breath slope value based on the first thoracic impedance waveform; comparing the first breath slope value to a first threshold slope value of the patient; and based on the comparison, providing an indication of respiratory function of the patient.

In another example, the disclosure is directed to system for monitoring respiratory function; the system including an impedance sensor, the impedance sensor configured to collect a first thoracic impedance waveform for at least one breath of a patient; a memory configured to store a first threshold slope value; and a processor comprising a signal analyzer configured to determine an first breath slope value based on the impedance waveform, compare the first breath slope value to a first threshold slope value, and based on the comparison, provide an indication of respiratory function of the patient.

In another example, the disclosure is directed to a computer-readable medium containing instructions. The instructions cause a programmable processor to receive a first thoracic impedance waveform for a at least one breath of a patient; determine a first breath slope value based on the first thoracic impedance waveform; compare the first breath slope value to a first threshold slope value; and based on the comparison, provide an indication of respiratory function of the patient.

In another example, a system includes means for receiving a first thoracic impedance waveform for at least one breath of a patient; means for determining a first breath slope value based on the first thoracic impedance waveform; means for comparing the first breath slope value to a first threshold slope value of the patient; and means for providing, based on the comparison, an indication of respiratory function of the patient.

Examples of this disclosure involve monitoring an impedance signal using a subcutaneous implantable medical device (IMD). In general, an impedance signal may be able to provide diagnostic information about multiple physiologic parameters including heart rate, cardiac function, respiratory rate, respiratory breathing anomalies and global fluid status. For example, respiratory anomalies such as obstructive sleep apnea (OSA), central sleep apnea (CSA), Cheyne-Stokes respiration or respiratory patterns associated with pulmonary edema may be detected with a subcutaneous impedance sensor placed in the thorax that can detect changes in respiratory rate, current respiratory rate, respiratory tidal volume, and characteristics of the patient's inspiration and expiration, including the amount of effort need to breathe.

In various examples consistent with the present disclosure, impedance waveforms may be monitored at specific times and under specific conditions. These conditions may be considered activation events. Limiting the collection of impedance waveforms to specific times and conditions allows for the system to better control for noise in the impedance waveform from outside influences. For example, if the impedance waveform is collected when a patient is sleeping, the impedance waveform is less likely to include noise resulting from the patient's movement. In addition, any increase in respiratory rate is less likely to have occurred because of the patient's activity level. Additionally, collecting the impedance waveform under specific conditions allows for the impedance waveform to be compared to a baseline impedance waveform collected under similar conditions.

In some examples, it may be desirable to collect impedance waveforms under multiple different sets of conditions. Therefore, in some examples, impedance waveforms may be collected in response to more than one activation event. For example, a physician or other clinician may be interested in monitoring for not only change in a patient's condition while sleeping over time, but also while the patient is awake. In such examples, the IMD may collect impedance waveforms in response to two or more activation events. The first activation event may be a time or portion of the day, e.g., between 10 pm and 5 am, and when an accelerometer or other posture sensor indicates that the patient 14 is within a posture or range of postures, e.g., lying flat. A second activation event may be between 10 am and 5 pm, when an accelerometer or other activity and posture sensor indicates that the patient has been sitting upright with an activity count below a predetermined level. Different baseline values may be stored for each of the activation events.

In general, the IMD or other computing device which analyzes the impedance waveform may store one or more baseline impedance waveforms, or one or more baseline values that are derivable from a baseline impedance waveform, such as one or more values of inspiration slope, expiration slope, inspiration area under the curve or expiration area under the curve. In some examples, the collection of these baseline impedance waveforms may be in response to the same activation events as are used to collect the impedance waveforms used to monitor the patient's respiratory function. In some examples, the impedance waveforms may be collected under controlled conditions where the physician or other clinician can make sure the baseline waveform does not include the presence of one or more respiratory anomalies.

The impedance waveform that is collected may be analyzed for a variety of characteristics that may help in the monitoring and diagnosis of a change in respiratory function of a patient. For example, the impedance waveform may be analyzed to determine respiratory rate, respiratory variability, area under the curve, inspiration slope, and mean impedance. In some examples, the area under the curve of the impedance waveform may be divided in to the area under the curve during inspiration and the area under the curve during expiration, i.e., the inspiration area and expiration area.

In some examples, the IMD may also monitor thoracic auscultations. The IMD may collect an acoustic waveform at approximately the same time that the impedance waveform is collected. The acoustic waveform may be analyzed to determine whether one or more abnormal breath sounds is present. For example, the IMD or other computing device may determine whether the acoustic waveform indicates the presence of rales, rhonchi, stridor, or wheezing, as examples.

Rales are small clicking, bubbling, or rattling sounds in the lung. Rales are believed to occur when air opens closed air spaces within the lungs. Rales can be further described as moist, dry, fine, and coarse. Rhonchi are sounds that resemble snoring. Rhonci occur when air is blocked or its passage through large airways of the lungs becomes turbulent. Stridor is a wheeze-like sound heard when a person breathes. Usually stridor is due to a blockage of airflow in the windpipe (trachea) or in the back of the throat. Wheezes are high-pitched sounds produced by narrowed airways. They can be heard when a person exhales.

In some examples, the IMD or other computing device may store a template acoustic waveform for each of these abnormal breathing conditions. In some examples, the IMD or other computing device may analyze the acoustic waveform to determine if increases have occurred in the low frequency or high frequency acoustic amplitude during inspiration and/or expiration. An increase in high frequency acoustic amplitude may indicate the presence of pulmonary edema, while an increase in low frequency acoustic amplitude may indicate the presence of OSA.

In general, the overall respiratory function may be determined by comparing the short term averages of various impedance parameter values to long term averages, or other baseline values, for the same impedance parameters. This may allow for the monitoring of a gradual decline in respiratory function that may be associated with compromised cardiac function. In some examples, the IMD monitors changes in respiratory impedance over time while the patient is awake or while the patient is asleep. In some examples, the IMD may also monitor for changes in the relative respiratory function of a patient between when the patient is awake and when the patient is asleep. This may be accomplished by comparing an impedance waveform collected in response a first activation event to an impedance waveform collected in response to a second activation event, and tracking the differences over time.

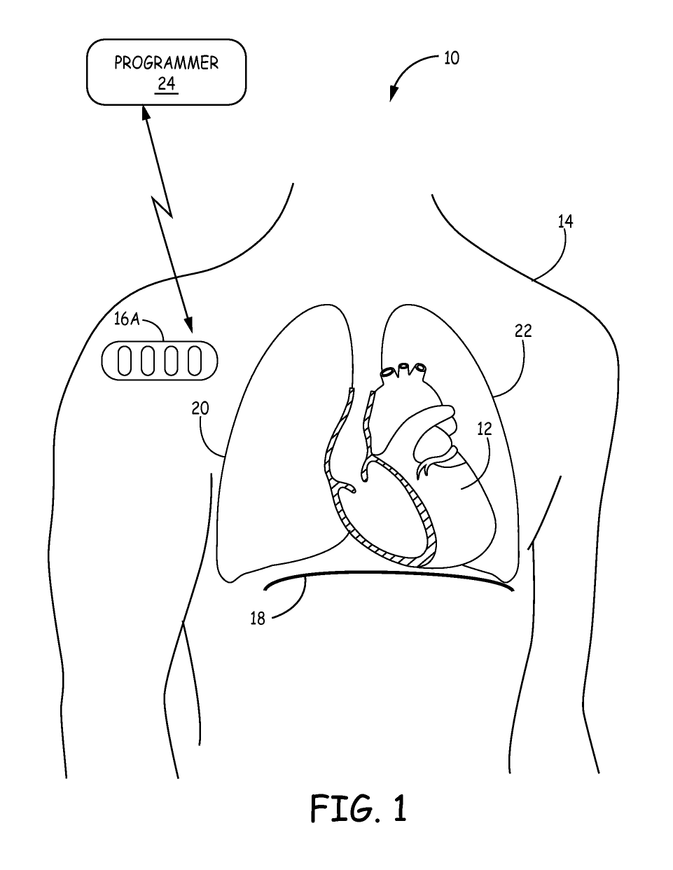

FIG. 1 is a conceptual diagram illustrating an example system 10 that measures physiological events associated with hemodynamic or respiratory function. In some examples, system 10 monitors both impedance and auscultation signals. Based at least in part on the impedance signal, system 10 determines whether patient 14 is experiencing a respiratory anomaly or a change in long-term patient health. In various examples, a respiratory anomaly detection sequence is initiated in response to a set of predetermined circumstances being met.

System 10 includes an implantable medical device (IMD) 16A which is optionally communicatively coupled to a programmer 24. IMD 16A senses electrical impedance signals representative of respiratory function. In some examples, IMD 16A also senses acoustic signals representative of respiratory function. IMD 16A may include a plurality of electrodes located on the housing of the IMD.

IMD 16A may monitor impedance of a current path that is between electrodes. In some examples, the current pathway traverses the heart, the lungs, and the thorax. The application of current results in the establishment of an electric field in the vicinity of the stimulation. The strength of the field dissipates with distance between the electrodes and surrounding the electrodes. In some examples, a second pair of electrodes may be used to sense changes in the established electrical field resulting from physiological impedance changes of tissues within the field. In other examples, the same electrodes used for stimulation may be used for sensing. In some examples, one sensing and one stimulating electrode can be used. The stronger the relative stimulation field intensity in a given sensed geometric region, the more sensitive the resultant impedance measurement to local impedance changes. It has been determined that the impedance of such a path is correlated with and varies in substantially the same manner as the pressure within lungs 20 and 22 or trachea (not shown). Thus, the impedance between electrodes may be monitored in order to determine characteristics of the breathing of patient 14, such as respiratory rate and tidal volume. Variations in these characteristics over time may indicate a change in respiratory function.

In some examples, programmer 24 takes the form of a handheld computing device, computer workstation or networked computing device that includes a user interface for presenting information to and receiving input from a user. A user, such as a physician, technician, surgeon, electro-physiologist, or other clinician, may interact with programmer 24 to retrieve physiological or diagnostic information from IMD 16A. A user may also interact with programmer 24 to program IMD 16A, e.g., select values for operational parameters of the IMD or initiate a phrenic nerve stimulation detection sequence.

IMD 16A and programmer 24 may communicate via wireless communication using any techniques known in the art. Examples of communication techniques may include, for example, low frequency or radiofrequency (RF) telemetry. Other techniques are also contemplated. In some examples, programmer 24 may include a programming head that may be placed proximate to the patient's body near the IMD 16A implant site in order to improve the quality or security of communication between IMD 16A and programmer 24. In other examples, programmer 24 may be located remotely from IMD 16A, and communicate with IMD 16A via a network.

Techniques for detecting respiratory function are primarily described herein as being performed by an IMD, such IMD 16A, e.g., by a processor and/or other component(s) of the IMD. In other examples, some or all of the functions ascribed to IMDs or a processor or component thereof may be performed by one or more other devices such as programmer 24, or a processor or other component thereof.

For example, IMD 16A may process impedance and/or auscultation signals to determine whether patient 14 has had a change in respiratory function. Alternatively, programmer 24 may process impedance and/or auscultation signals received from IMD 16A to determine whether a change in respiratory function. Furthermore, although described herein with respect to an IMD, in other examples, the techniques described herein may be performed or implemented in an external medical device, which may be coupled to a patient vie percutaneous or transcutaneous leads. In some examples, various functions of IMD 16 may be carried out by multiple IMDs in communication with one another.

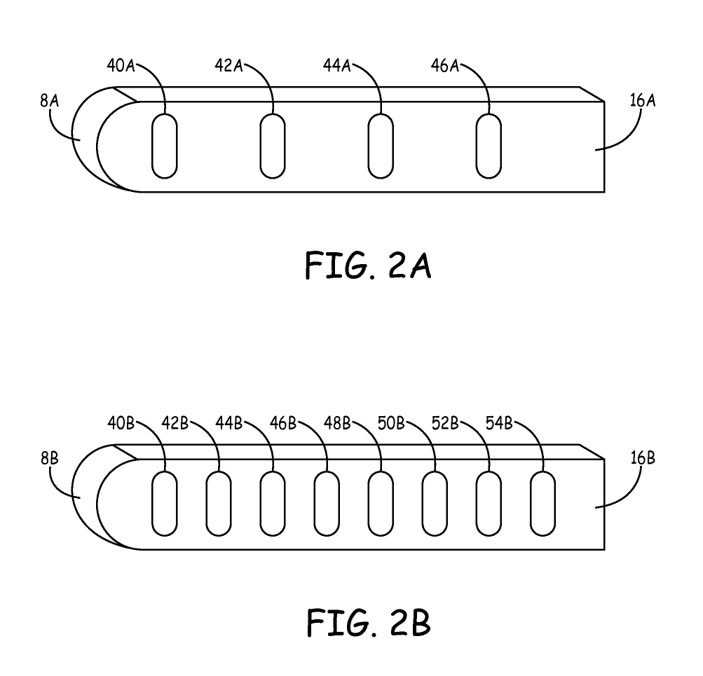

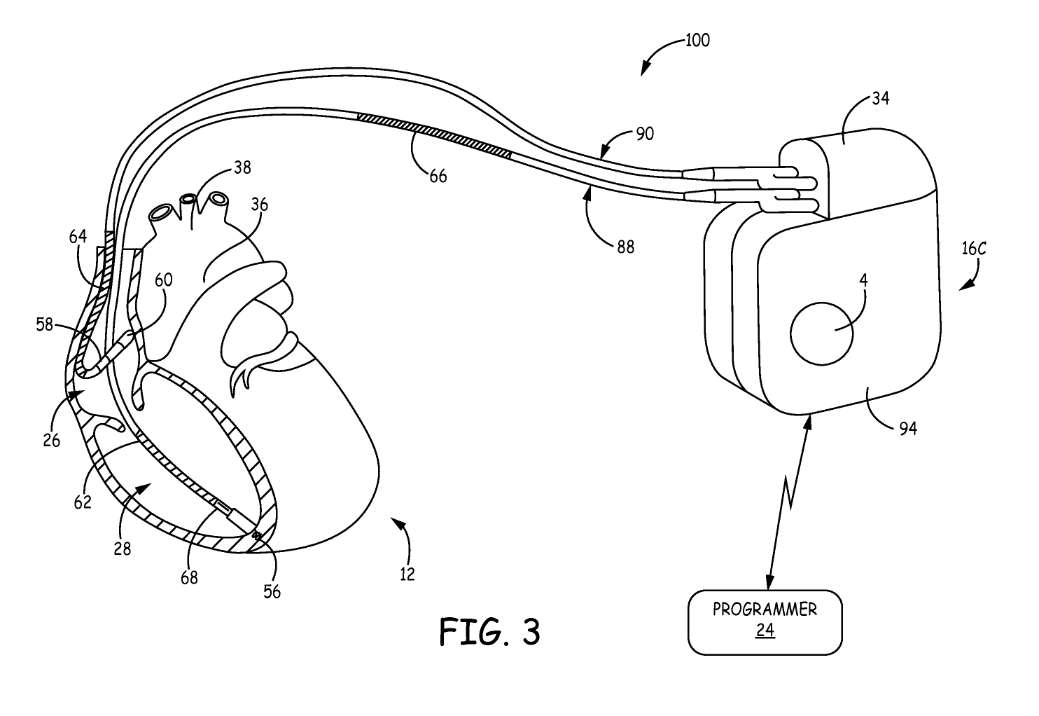

FIG. 2A is a conceptual diagram illustrating IMD 16A of therapy system 10A in greater detail. FIG. 2B is a conceptual diagram illustrating another example IMD 16B. IMDs 16A and 16B, as well as IMD 16C (FIG. 3), may be collectively referred to as “IMDs 16, “and individually referred to generally as an “IMD 16.”

In some examples, as illustrated In FIGS. 2A and 2B, an IMD includes a plurality of housing electrodes which may be formed integrally with an outer surface of a hermetically sealed housing of the IMD, or otherwise coupled to housing 8. In the example of FIG. 2A, IMD 16A may include four electrodes on a first side (shown) and an additional four electrodes on a second side (not shown). Alternatively, IMD 16A may include only 4 electrodes. For example, IMD 16A may include electrodes 40A, 42A, 44A, and 46A on one side of housing 8A. Housing electrodes 40A, 42A, 44A, and 46A may be defined by uninsulated portions of a portion, e.g., an outward facing portion, of housing 8A of IMD 16A. IMD 16A may generate an electrical field between a pair of electrodes. For example, electrodes 42A and 46B may generate an electrical from which impedance measurements are made.

In the example of FIG. 2B, IMD 16B includes an array of eight electrodes, 40B, 42B, 44B, 46B, 48B, 50B, 52B and 54B. IMD 16B may be implanted within patient 14, and may generally function similarly to IMD 16A as shown and described with respect to FIG. 1 As was the case with IMD 16A, housing electrodes 40B, 42B, 44B, 46B, 48B, 50B, 52B and 54B of IMD 16B may defined by uninsulated portions of an outward facing portion of housing 8B of IMD 16B.

Impedance may be measured along a path between any two of the housing electrodes of IMDs 16A or 16B. In some examples, multiple pairs of electrodes may be selected to generate multiple electrical fields. The electrodes may be selected such that the multiple electrical fields are substantially homogenous or uniform over the thoracic region of interest.

For example, electrodes 40B and 54B may be used to stimulate the tissue (by injecting current from one to the other) and electrodes 44B and 50B may be used to measure the impedance of the resulting current. The combined electrical field may be substantially homogenous over portions of interest of patient tissue, e.g., in the lungs 20 and 22, heart 12 and relative blood volume of the thorax, thereby resulting in a more accurate estimation than if only a single pair of electrodes were used to generate a single electrical field. If four electrodes are used, current injection may be between the outer two electrodes and voltage sensing may be between the inner two electrodes. In examples with only two electrodes being used, both electrodes are used for current injection and voltage sensing.

In additional examples, multiple pairs of measurement electrodes may be selected to filter out “noise” resulting from the electrical field travel through regions that are not of interest. For example, electrodes 42B and 46B may generate a first electrical field across portions of right ventricle 28 and left ventricle 32 and a first impedance may be measured by electrodes 40B and 44B. In addition, electrodes 42B and 52B may generate a second electrical field across portions of heart 12 and a second impedance may be measured by electrodes 42B and 52B. The first and second impedances may be subtracted or otherwise processed to determine the impedance associated the lungs 20 and 22 and diaphragm 18. In this manner, multiple measurement electrodes may be used to filter out “noise” within a measured signal and thereby provide a more robust determination of impedance values of a path.

In the examples of FIGS. 2A and 2B, IMDs 16 may be implanted in patient 14 in numerous places. In some examples, an IMD 16 is a subcutaneous device placed within the thorax. In one example, an IMD 16 is approximately the size and shape of the Reveal® Plus implantable loop recorder, by Medtronic, Inc. of Minneapolis, Minn.

FIG. 3 is a conceptual diagram illustrating another example of a therapy system 100, which is similar to therapy system 10 of FIGS. 1-2B, but includes two leads 88 and 90, and may provide stimulation to the heart 12 in addition to monitoring respiratory function. Leads 88 and 90 are implanted within right ventricle 28 and right atrium 26, respectively. Leads 88 and 90 may be electrically coupled to a stimulation generator and sensing module of IMD 16C via connector block 34. In some examples, proximal ends of leads 88 and 90 may include electrical contacts that electrically couple to respective electrical contacts within connector block 34 of IMD 16C. In addition, in some examples, leads 88 and 90 may be mechanically coupled to connector block 34 with the aid of set screws, connection pins, snap connectors, or other suitable mechanical coupling mechanism.

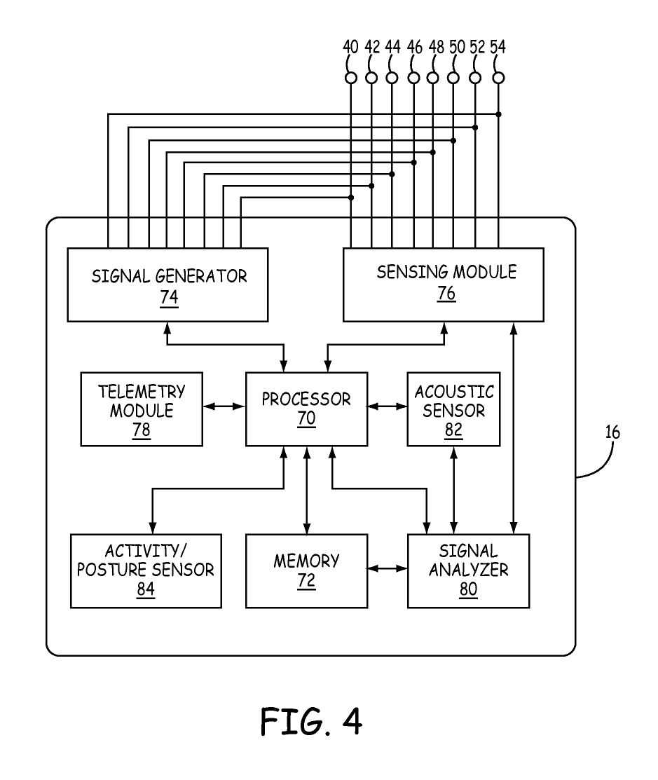

In some examples, as shown in FIG. 3, IMD 16C includes one or more housing electrodes, such as housing electrode 4, which may be formed integrally with an outer housing of hermitically sealed housing 94 of IMD 16C or otherwise coupled to housing 94. In some examples, housing electrode 4 may be defined by an uninsulated portion of housing 94, such as uninsulated portion of an outward facing portion of housing 94 of IMD 16C. Other division between insulated and uninsulated portions of housing 94 may be employed to define two or more housing electrodes. In some examples, housing electrode 4 comprises substantially all of housing 4. As described, in further detail with reference to FIG. 4, housing 94 may enclose a signal generator that generates therapeutic stimulation, such as cardiac pacing pulses and defibrillation shocks, as well as a sensing module for monitoring the rhythm of heart 12.

Each of leads 88 and 90 includes an elongated insulative lead body, which may carry a number of concentric coiled conductors separated from one another by tubular insulative sheaths. Bipolar electrodes 68 and 56 are located adjacent to a distal end of lead 88 in right ventricle 28. In addition bipolar electrodes 58 and 60 are located adjacent to a distal end of lead 90 in right atrium 26. There are no electrodes located in left atrium 36, but other examples may include electrodes in left atrium 36. Furthermore, other examples may include electrodes in other locations, such as the aorta or vena cava, or epicardial or extracardial electrodes proximate to any of the chamber or vessels described herein.

Additionally, lead 88 includes electrodes 62 and 66, which may take the form of a coil, as in the example of FIG. 3. In this manner, electrodes 62 and 4, or electrodes 26 and 66 for example, may be used to generate an electrical field and to measure impedance values across lungs 20 and 22 and heart 12 (FIG. 1).

IMD 16C may sense electrical signals attendant to the depolarization and repolarization of heart 12 via electrodes 56, 58, 60, 68, 4, 62, 64, and 66. The electrical signals are conducted to IMD 16C from the electrodes via the respective leads 88 and 90 or, in the case of housing electrode 4 a conductor coupled to housing electrode 4. IMD 16C may sense such electrical signals via any bipolar combination of electrodes 56, 58, 60, 68, 62, 64, and 66. Furthermore, any of the electrodes 56, 58, 60, 68, 62, 64, and 66 may be used for unipolar sensing in combination with housing electrode 4.

In some examples, IMD 16C delivers pacing pulses via bipolar combinations of electrodes 56, 58, 60, and 68 to produce depolarization of cardiac tissue of heart 12. In some examples, IMD 16C delivers pacing pulses via any of electrodes 56, 58, 60, and 68 in combination with housing electrode 4 in a unipolar configuration. Furthermore, IMD 16C may deliver defibrillation pulses to heart 12 via any combination of elongated electrodes 62, 64, 66, and housing electrode 4. Electrodes 4, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

Any combination of electrodes 4, 56, 58, 60, 62, 64, 66 and 68 may be used for measuring impedance in accordance with the techniques of this disclosure. In some examples an electrode pair may be selected to generate an electrical field and to measure the impedance of the resulting current. In some examples, multiple electrodes pairs may be selected to generate multiple electrical field as discussed above with respect to FIGS. 2A and 2B.

Therapy system 100 shown in FIG. 3 may also be useful for providing defibrillation and pacing pulses to heart 12. System 100 may also determine impedance values in accordance with the techniques described herein. Moreover, system 100 may determine changes in the impedance values over time to determine changes in respiratory function and, more generally, hemodynamic status, as discussed herein.

The configuration of therapy systems 10 and 100 illustrated in FIGS. 1-3 are merely examples. It should be understood that various other electrode and lead configurations for measuring impedance are within the scope of this disclosure. For example, IMD 16C need not be implanted within patient 14. For examples in which IMD 16C is not implanted in patient 14, IMD 16C may deliver defibrillation pulses and other therapies to heart 12 via percutaneous leads that extend through the skin of patient 14 to a variety of positions within or outside of heart 12.

FIG. 4 is a block diagram illustrating an example configuration of an IMD 16. Any of IMDs 16 illustrated in FIGS. 1-3 may be configured the same as or substantially similarly to the example configuration of IMD 16 illustrated in FIG. 4.

In the illustrated example, IMD 16 include a processor 70, memory 72, signal generator 74, sensing module 76, telemetry module 78, a signal analyzer 80, an acoustic sensor 82, an activity/posture sensor 84, and a power source 86. Memory 72 includes computer-readable instructions, that, when executed by processor 70, cause IMD 16 and processor 70 to perform various functions attributed to IMD 16 and processor 70 herein. Memory 72 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 70 may include any one or more of a microprocessor, a controller, a digital signal processor (DSP), an application specific integrated circuit (ASIC), a field-programmable gate array (FPGA), or equivalent discrete or analog logic circuitry. In some examples, processor 70 may include multiple components, such as any combination of one or more microprocessors, one or more controllers, one or more DSPs, one or more ASICs, or one or more FPGAs, as well as other discrete or integrated logic circuitry. The functions attributed to processor 70 herein may be embodied as software, firmware, hardware or any combination thereof. Generally, processor 70 controls signal generator 74 to deliver signals to generate one or more electrical fields between at least two electrodes for impedance measurements.

Stimulation generator 74 may include a switch module and processor 70 may use the switch module to select, e.g., via a data/address bus, which of the available electrodes are used to deliver defibrillation pulses or pacing pulses. Processor 70 may also control which of electrodes 40, 42, 44, 46, 48, 50, 52, and 54, is coupled to signal generator 84 for impedance measurements, e.g., via the switch module. The switch module may include a switch array, switch matrix, multiplexer, or any other type of switching device suitable to selectively couple a signal to selected electrodes.

Telemetry module 78 includes any suitable hardware, firmware, software or any combination thereof for communicating with another device, such as programmer 24 (FIG. 1). Under the control of processor 70, telemetry module 78 may receive downlink telemetry from and send uplink telemetry to programmer 24 with the aid of an antenna, which may be internal and/or external. Processor 70 may provide the data to be uplinked to programmer 24 and the control signals for the telemetry circuit within telemetry module 78, e.g., via an address/data bus. In some examples, telemetry module 78 may provide received data to processor 70 via a multiplexer (not shown).

In some examples, processor 70 may transmit impedance signals produced by amplifier circuits within electrical sensing module 76 to programmer 24. Programmer 24 may interrogate IMD 16 to receive the impedance signals. Processor 70 may store impedance signals within memory 72, and retrieve stored impedance signals from memory 72.

In the example in FIG. 4 (e.g., to detect the respiratory function), IMD 16 also includes acoustic sensor 82. Acoustic sensor 82 generates an electrical signal based on sensed acoustics or vibrations originating from movement of diaphragm 18 or lungs 20 and 22, or breath sounds, for example. In some examples, acoustic sensor 82 may comprise more than one sensor. For example, the acoustic sensor 82 may include multiple individual sensors. In some examples, acoustic sensor 82 is an acoustic sensor, such as an accelerometer, microphone, or piezoelectric device. The acoustic sensor picks up sounds resulting from the activation of the diaphragm in addition to breath sounds such as rales, rhonchi, stridor, and wheezing.

In the illustrated example of FIG. 4, acoustic sensor 82 is enclosed within the housing of IMD 16. In some examples, acoustic sensor 82 may be formed integrally with or on an outer surface the housing of IMD 16. In some examples, acoustic sensor 82 is located on one or more leads that are coupled to IMD 16 (shown in FIG. 4) or may be implemented in a remote sensor that wirelessly communicates with IMD 16. In such cases, heart sounds sensor 82 may be electrically or wirelessly coupled to circuitry contained within the housing of IMD 16. In some examples, a remote acoustic sensor 82 may be wirelessly connected to programmer 24.

Signal analyzer 80 receives the electrical signal generated by sensing module 76 and/or acoustic sensor 82. In one example, signal analyzer 80 may process the sensor signal generated by acoustic sensor 82 to detect occurrences of abnormal breathing sounds. In some examples, signal analyzer 80 may process the impedance signal generated by sensing module 76 to detect characteristics of respiratory function such as respiratory rate, inspiration and expiration slope, and tidal volume. In some examples, processes the acoustic sensor signal and the impedance signal to remove extraneous information do to heart functions. For examples, the acoustic sensor signal may be processed remove heart sounds, and the impedance signal may be processed to remove changes in impedance due to cardiac function.

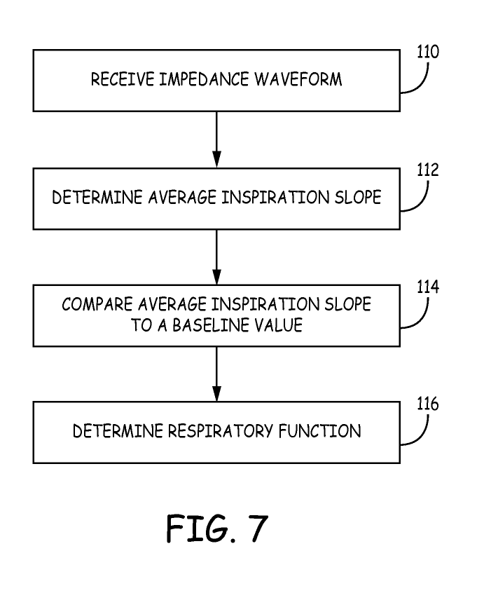

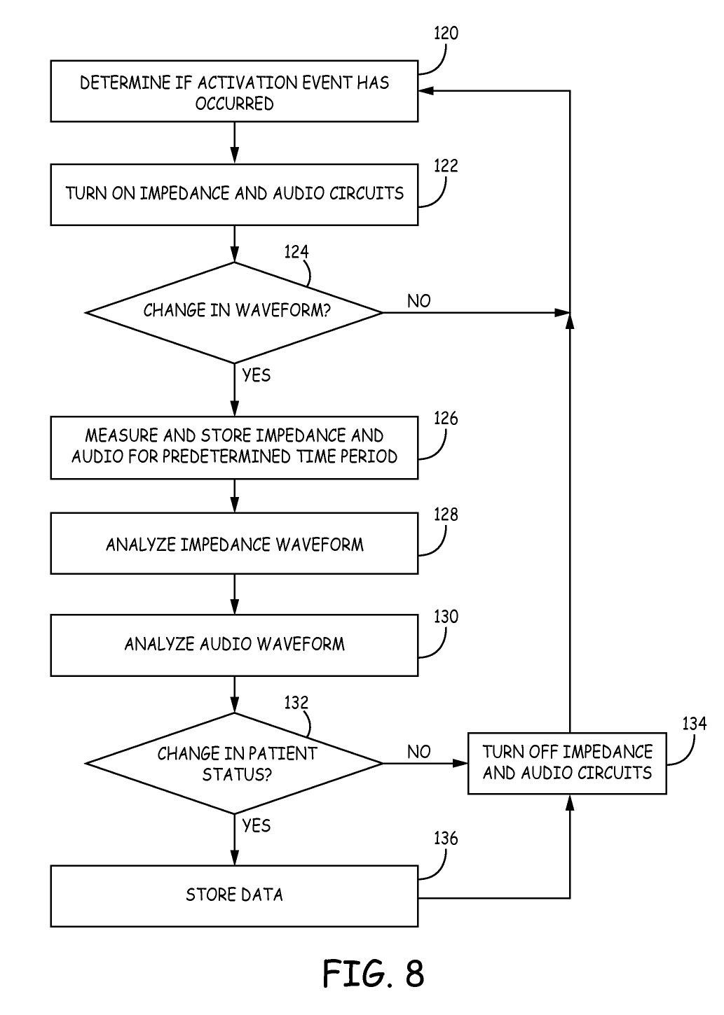

In some examples, an unprocessed impedance signal is provided to processor 70 by sensing module 76 for impedance information extraction. In some examples, the impedance information may also be extracted from the EGM signal by the signal analyzer 80. Examples of the operation of signal analyzer 80 and processor 70 in accordance with these example methods are described in greater detail with respect to FIGS. 7-8.

A respiratory function indication may be outputted from signal analyzer 80 to processor 70. In some examples, respiratory function indications may be determined by processor 70. The processor 70 may determine whether a particular respiratory anomaly or a change in patient status has occurred based on the information received from signal analyzer 80. In some examples, processor 70 may store information regarding a detected respiratory anomaly or change in patient status in memory 72. In some examples, processor 70 may transmit information regarding the detected respiratory anomaly or change in patient status to programmer 24 via telemetry module 78.

In various examples one or more of the functions attributed to signal analyzer 80 may be performed by processor 70. In some examples, signal analyzer 80 may be implemented as hardware, software, or some combination thereof. For example, the functions of signal analyzer 80 described herein may be implemented in a software process executed by processor 70.

Processor 70 may initiate a respiratory function detection sequence in response to detecting an activation event. In some examples, processor 70 may receive an activation signal from programmer 24 via telemetry module 78, which may be the activation event, before initiating respiratory function detection. In some examples, the activation event may be one or more of an activity/posture detected via activity/posture sensor 84, signal analyzer 80, memory 72, and sensing module 76. In some examples, processor 70 may initiate respiratory function detection at a given time. For example, memory 72 may provide a program to processor 70 wherein respiratory function detection occurs every day at a predetermined time. In such cases the activation event is the time of day.

In other examples, processor 70 may initiate respiratory function detection during a predetermined time range when predefined parameters are met. For example, processor 70 may initiate respiratory function detection between 10 p.m. and 5 a.m. when an activation event, such as activity/posture sensor 84 indicating that patient 12 is lying down, occurs. In some specific examples, processor 70 may initiate respiratory function detection in response to an activation event such as an indication from the activity/posture sensor 84 that patient 12 is lying on his or her left side is received. In some examples, such as the example of FIG. 3, processor 70 may initiate a respiratory detection sequence based on an activation event such as one or more pacing parameters changing.

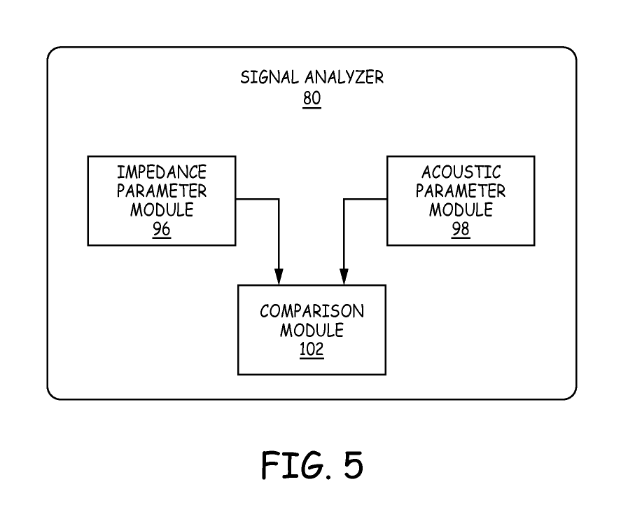

FIG. 5 is a block diagram illustrating an example configuration of signal analyzer 80. In general, Signal analyzer 80 may receive a plurality of impedance values from electrical sensing module 76, the values indicating the impedance of a path between electrodes, such as electrodes 40 and 44, over time. Signal analyzer 80 may also receive instructions for processor 70. Signal analyzer 80 may also read and store data and instructions in memory 72.

Impedance parameter module 96 may determine an impedance parameter value based on the impedance values. Signal analyzer 80 may store the impedance parameter value in memory 72. In one example, signal analyzer 80 may store a plurality of impedance parameter values in memory 72. In some examples, one or more impedance parameter values may be stored over time.

In one example, signal analyzer 80 may additionally store identifying information for the impedance parameter value. For example, in one example, processor 70 may determine a particular activation event has occurred to trigger collection of impedance signals. Identifying information may include, for example, a sequence number that enumerates each time a particular activation occurs. Impedance parameter module 96 may determine an impedance parameter value for based on the impedance values. In some examples, impedance parameter module 96 may determine when a breath has occurred. The breath may be detected based on local minimum impedance values. In some examples, the change from inspiration to expiration may be detected based on a local maximum between adjacent local minimums.

For example, impedance parameter module 96 may determine an average or mean value of the impedance values over a predetermined time period. As another example, impedance parameter module 94 may identify a particular value among impedance values obtained over a respiratory cycle, such as a maximum amplitude or a minimum amplitude. As another example, impedance parameter module 96 may determine respiratory rate based on impedance values. As another example, impedance parameter module 96 may determine a respiratory tidal volume based on the impedance values.

As another example, impedance parameter module 96 may determine respiration area under the curve. The area under the curve may be determined as an integral of the impedance values between two points in time. For example, area under the curve during inspiration may be determined between a local minimum amplitude and a local maximum amplitude. Area under the curve during expiration may be determined between the local maximum and a second local minimum. In some examples, the area under the curve is determined between two local minimums and separated into inspiration area under the curve and expiration area under the curve based on the location of a local maximum between the two minimums.

In some examples, impedance parameter module 96 may determine inspiration slope. As another example, impedance parameter module 96 may determine expiration slope. The inspiration and expiration slopes may be indicators of respiratory compliance. If pulmonary edema develops, lung compliance may decrease, resulting in a decrease in expiration slope, for example. Impedance parameter module 96 may also determine respective values for each of a plurality of different impedance parameters, e.g. a mean and a range, or a mean, a derivative, an integral, and an amplitude, or some other combination of impedance parameter values, for each respiratory cycle. Impedance parameter module 94 may store each of the determined impedance parameter values in memory 72.

Acoustic parameter module 98 may determine characteristics of an acoustic signal based on the acoustic signal from acoustic sensor 82. Signal analyzer 80 may store values for acoustic characteristics in memory 72. In one example, signal analyzer 80 may store a plurality of acoustic characteristics in memory 72. In some examples, one or more acoustic characteristics may be stored in memory 72 over time. In some examples, the acoustic characteristics may be associated with impedance parameter values also stored in memory 72. For example, acoustic characteristics and impedance parameter values may be associated based on time. As an example, acoustic parameter module 98 may determine inspiration duration for each respiratory cycle. As another example, acoustic parameter module 98 may determine expiration duration for reach respiratory cycle. As another example, acoustic parameter module 98 may determine whether the acoustic signal indicates the presence of abnormal breath sounds such as rales, rhonci, stridor or wheezing.

Comparison module 102 may determine that a change in patient status has occurred by, for example, determining a difference or ratio between a current impedance parameter value and a baseline impedance parameter value. In some examples, the baseline impedance parameter value may be an impedance parameter value collected at a time near implantation of IMD 16.

Signal analyzer 80 may diagnose a respiratory anomaly based on one or more impedance parameter values identified by impedance parameter module 96. In some examples, comparison module 102 may compare impedance parameter values determined by impedance parameter module 96 to baseline impedance parameter values stored in memory 72. In some examples, the baseline impedance parameter values are impedance parameter values collected and stored during an initial visit with a clinician or other care provider after the implantation of IMD 16. In some examples, the baseline impedance parameter values were collected in response to the same type of activation event as the current impedance parameter values. In some examples, the baseline impedance parameter values are collected and stored do not include a respiratory anomaly. In another example, a set of baseline parameter values may correspond to a particular respiratory anomaly. Comparison module 102 may compare impedance parameter values determined by impedance parameter module 96 to the stored impedance parameter values. In some examples, based on the comparison, signal analyzer 80 may determine whether a respiratory anomaly has occurred. In some examples, signal analyzer 80 may determine whether a change in patient status has occurred.

In some examples, comparison module 102 may compare impedance parameter values to acoustic characteristics determined by acoustic parameter module 98. In some examples, the acoustic characteristics may be used to confirm a diagnosis of a respiratory anomaly or a change in patient status. In some examples, a diagnosis of a respiratory anomaly or a change in patient status may be based on a combination of information from the impedance parameter values and the acoustic characteristics.

Although examples in which signal analyzer 80 and its components, e.g., comparison module 102, are implemented within IMD 16 are described herein, in other examples, signal analyzer or any of its components may be implemented in another device, e.g., external to IMD 16. In one such example, programmer 24 may include a comparison module 102 to perform the comparison of two or more impedance parameter values. Programmer 24 may retrieve the impedance parameter values from IMD 16 through, e.g., telemetry module 88. Programmer 24 may then perform the comparison to determine whether the impedance parameter values indicate a change in respiratory function or patient status. Furthermore, in some examples, programmer 24 may include impedance parameter module 96, and determine impedance parameter values based on values that indicate impedance measured by IMD 16.

An indication of a respiratory anomaly or change in patient status may be output from signal analyzer 80 to processor 70. In some examples, the impedance parameter values are output to the processor 70. The processor 70 may determine whether a change in respiratory function has occurred based on the information received from signal analyzer 80. In some examples, processor may adjust stimulation provided by signal generator 74 based on the impedance-based information received.

In various examples one or more of the functions attributed to signal analyzer 80 may be performed by processor 70. In some examples, signal analyzer 80 may be implemented as hardware, software, or some combination thereof. For example, the functions of signal analyzer 80 described herein may be implemented in a software process executed by processor 70.

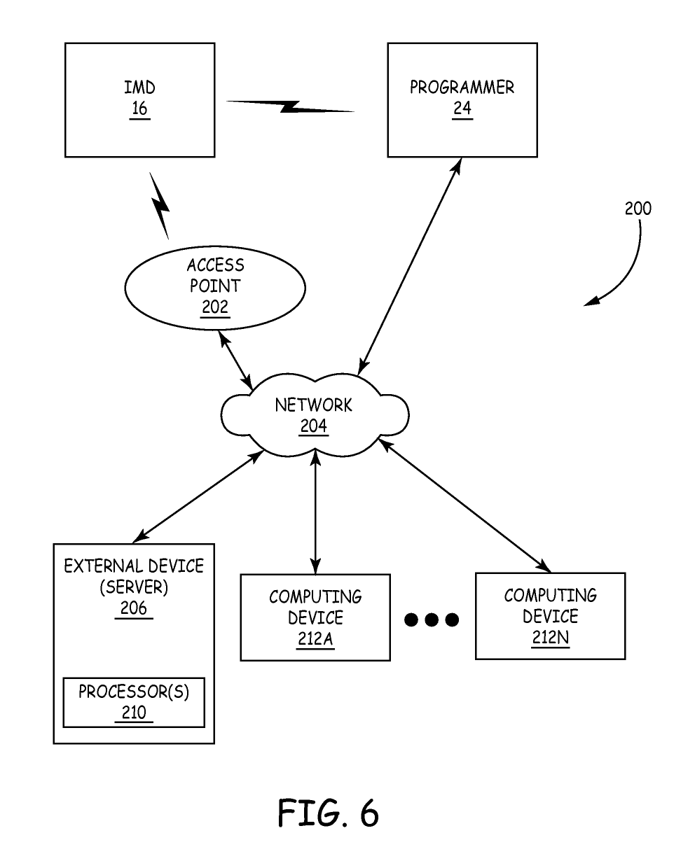

FIG. 6 is a block diagram illustrating an example system 200 that includes an external device, such as a server 206, and one or more computing devices 212A-212N that are coupled to the IMD 16 and programmer 24 shown in FIG. 1 via a network 204. Network 204 may be generally used to transmit diagnostic information (e.g., the occurrence of a change in respiratory function) from an IMD 16 to a remote external computing device. In some examples, the impedance signals and/or the acoustic signals generated by a sensing module 76 and/or acoustic sensor 82 of an IMD 16 may be may be transmitted to an external device for processing, e.g., the external device may include signal analyzer 80 or otherwise implement some or all of the functionality attributed to signal analyzer 80 herein.

In some examples, the IMD 16 transmits information when impedance (and in some cases acoustic) sensing is active. In some examples, an activation event may result in the initiation of a respiratory function detection sequence in which the sensing is activated. In some examples, IMD 16 may also transmit information to the external device regarding the activation event that triggered the respiratory function detection sequence to the remote external computing device. For example, the IMD may transmit the time, position and activity level of the patient to external server 206, computing device 212 or programmer 24.

In some examples, the information transmitted by IMD 16 may allow a clinician or other healthcare professional to monitor patient 14 remotely. In some examples, IMD 16 may use its telemetry module 78 to communicate with programmer 24 via a first wireless connection, and to communicate with an access point 202 via a second wireless connection, e.g., at different times. In the example of FIG. 6, access point 202, programmer 24, server 206, and computing devices 212A-212N are interconnected, and able to communicate with each other, through network 204. In some cases, one or more of access point 202, programmer 24, server 206, and computing devices 212A-212 N may be coupled to network 204 via one or more wireless connections. IMD 16, programmer 24, server 206, and computing devices 212A-212N may each comprise one or more processors, such as one or more microprocessors, DSPs, ASICs, FPGAs, programmable logic circuitry, or the like, that may perform various functions and operations, such as those described herein.

Access point 202 may comprise a device that connects to network 204 via any of a variety of connections, such as telephone dial-up, digital subscriber line (DSL), or cable modem connections. In other examples, access point 202 may be coupled to network 204 through different forms of connections, including wired or wireless connections. In some examples, access point 202 may be co-located with patient 14 and may comprise one or more programming units and/or computing devices (e.g., one or more monitoring units) that may perform various functions and operations described herein. For example, access point 202 may include a home-monitoring unit that is co-located with patient 14 and that may monitor the activity of IMD 16. In some examples, server 206 or computing devices 212 may control or perform any of the various functions or operations described herein, e.g., determine, based on impedance, whether the patient's respiratory function is compromised or has changed over time.

In some cases, server 206 may be configured to provide a secure storage site for archival of diagnostic information (e.g., occurrence of phrenic nerve stimulation and attendant circumstances such as pacing parameters) that has been collected and generated from IMD 16 and/or programmer 24. Network 204 may comprise a local area network, wide area network, or global network, such as the Internet. In some cases, programmer 24 or server 206 may assemble respiratory function information in web pages or other documents for viewing by and trained professionals, such as clinicians, via viewing terminals associated with computing devices 212. The system of FIG. 6 may be implemented, in some aspects, with general network technology and functionality similar to that provided by the Medtronic CareLink® Network developed by Medtronic, Inc., of Minneapolis, Minn.

In the example of FIG. 6, external server 206 may receive impedance signal information from IMD 16 via network 204. Based on the impedance signal information received, processor(s) 210 may perform one or more of the functions described herein with respect to signal analyzer 80 and processor 70. In some examples, an external device such as server 206 or computing devices 212 may provide an activation signal to IMD 16 via network 204. In response to the activation signal, IMD 16 may initiate a respiratory function detection sequence consistent with one or more of the methods described herein with respect to FIGS. 7-8, for example. In some examples, impedance and/or acoustic signals are transmitted to the external device that sent the activation signal. The external device, such as server 206 processes the signals to determine whether a change in respiratory function has occurred.

FIG. 7 is a flow chart illustrating an example mode of operation of IMD 16 for assessing respiratory function using impedance signals. Although discussed with respect to IMD 16, processor 70 and signal analyzer 80, one of skill in the art would understand the method of FIG. 7 may be implemented by one or more processors, including external processor 210 or a processor in programmer 24.

IMD 16 receives an impedance waveform (110). In some examples, sensing module 76 detects the impedance waveform. The impedance waveform may include impedance information for a predetermined length of time or number of breaths. For example, the impedance waveform may be approximately 5 minutes in length. In some examples, the impedance waveform is received in response to an activation event. IMD 16 then determines an average inspiration slope (112) for the impedance waveform. In some examples, signal analyzer 80 determines the average inspiration slope (112). In some examples, signal analyzer 80 determines other impedance parameter values, including, for example, respiratory rate, respiratory variability, area under the curve, mean impedance, and expiration slope.

The average inspiration slope may be determined based on an approximation of a curve based on a discrete sampling of impedance values. The average inspiration slope may be a first derivative of the curve approximation between a determining beginning of inspiration and a determined beginning of expiration. In some examples, the beginning of inspiration may be determined based on a local minimum impedance value. The beginning of expiration may be determined based on a local maximum impedance value.

Signal analyzer 80 then compares the average inspiration slope to a baseline value (114) for inspiration slope. In some examples, the baseline value is an average value for inspiration slope obtained in response to the same activation event as the current impedance waveform. In some examples, the baseline value is associated with a particular patient state or level or respiratory function. In some examples, the baseline value may be a long term average of inspiration slopes for the patient.

Based at least in part on the comparison between the average inspiration slope for the breaths within the impedance waveform, IMD 16 determines respiratory function (116) for patient 14. In some examples, IMD 16 determines whether there has been a change in respiratory function. The change in respiratory function may be a respiratory anomaly such as CSA, OSA, or Cheyne-stokes. In some examples, the change in respiratory function may indicate a change in patient status. For example, the patient's overall breathing quality may have changed. In some examples, a worsening in respiratory function may indicate a worsening in cardiac function. In some examples, a classification of the current respiratory function may be based on Table A, below.

Clinical Conditions Table A (Impedance)

| | Respi- | | Area | | Decrease In |
|---|---|---|---|---|---|
| Clinical | ratory | Respiratory | Under | Inspiration | Mean |
| Condition | Rate | Variability | Curve | Slope | Impedance |
| Cheynne | Normal | High | Low/ | Increased | Low |
| Stokes | | | High | | |
| Central | Low | High | Low | Increased | Low |
| Sleep Apnea | | | | | |
| Obstructive | Low | Medium/ | Low | Decreased | Low |
| Sleep Apnea | | High | | | |
| Pulmonary | High | Low | High | Increased | High |
| Edema | | | | | |

As shown in Table A IMD 16 may diagnosis a change in respiratory function base on respiratory rate, respiratory variability, Area under the curve, inspiration slop and decrease in mean impedance. In general the measured impedance has both an oscillating component (quantified by peak to peak changes), and a dc or mean component. The mean is the net basal impedance of all the tissue in the field. The oscillatory component is due to change in the basal impedance due to respiration. The respiratory rate, respiratory variability, area under the curve and inspiration slope are determined from the oscillating component. However, the basal mean impedance provides inspiration about the patient's underlying steady state. If the net impedance of all the tissue in the field is high, this may indicate the presence of more fluid, and result in a diagnosis of pulmonary edema.

FIG. 8 is a flow chart illustrating an example mode of operation of IMD 16 for assessing respiratory function using impedance signals. Although discussed with respect to IMD 16, processor 70 and signal analyzer 80, one of skill in the art would understand that the method of FIG. 8 may be implemented by one or more processors, including external processor 210 or a processor in programmer 24.

According to the example of FIG. 8, processor 70 may determine if an activation event has occurred (120). Processor 70 may make this determination based at least in part on information from activity/posture sensor 84. In some examples, the activation event may include an indication that the patient is lying flat during a predetermined time range. In response to a determination that an activation event has occurred, processor 70 turns on the impedance and acoustic sensing circuits (122) of IMD 16. In some examples, this includes providing an electrical field generated signal generator 74 and sensing the electrical field using sensing module 76. In addition, acoustic sensor 82 is turned on. In some examples, impedance and acoustic signals are collected for a predetermined period of time after the circuits are turned on. In some examples, turning on the impedance and acoustic circuits may include storing the impedance and acoustic signals.

Signal analyzer 80 determines if there has been a change in the impedance or acoustic waveforms (124). In some examples, signal analyzer 80 determines if there has been a change in at least one of the waveforms. In some examples, signal analyzer 80 determines whether there has been a change in both waveforms. In one example, signal analyzer performs a real-time determination of whether a change in waveform has occurred, or may have occurred.

In some examples, signal analyzer 80 determines whether a change has occurred by comparing impedance parameter values of the impedance waveform to stored baseline values for the impedance parameters. In some examples, signal analyzer 80 also compares the acoustic waveform to a baseline acoustic waveform. For examples, signal analyzer 80 may be determine if there has been a change in respiratory rate based on the acoustic waveform. In other examples, a change in respiratory rate may be detected based on impedance signal. In one example, the difference in impedance parameter values or in acoustic characteristics relative to their baselines must be greater than a predetermined threshold in order to be considered a change in waveform.

In some examples, a change in acoustic characteristics may indicate the presence of rales, ronchi, stridor or wheezing. In response to a detected change in acoustic characteristics, the acoustic signal may be compared to a specific template for rales, ronchi, stridor, or wheezing to determine what type of breathing abnormality, if any, is present.

In some examples, signal analyzer compares values for impedance parameters determined for each breath, or for subsets of breaths, within the impedance waveform. This may be used to determine if a respiratory anomaly, such as a change in respiratory rate, has occurred within the timeframe that the impedance waveform and acoustic waveform were collected. If no change in impedance waveform and/or acoustic waveform has occurred, processor 70 resumes monitoring for the occurrence of an activation event. In some examples, a predetermined amount of time must pass before reactivation of the impedance and acoustic circuits in response to an activation event. For example, processor 70 may not reactivate the impedance and acoustic circuits until at least 5 minutes has passed.

In some examples, the impedance waveform may be compared to a template impedance waveform. If the difference, e.g., percentage, sum of differences, or the like, between the current impedance waveform and the template impedance waveform is greater than a predetermined threshold, signal analyzer 80 determines there has been a change in waveform. The collected acoustic waveform may also be compared to a template acoustic waveform. If the difference, e.g., percentage, sum of differences, or the like between the current acoustic waveform and the template acoustic waveform is greater than a predetermined threshold, signal analyzer 80 determines there has been a change in waveform.

In response to the determination that a change in waveform has occurred (124), IMD 16 measures, and stores, impedance and acoustic signals for a predetermined period of time (126). In some examples, the number of measurements collected may discrete signals at a higher frequency than during passive monitoring. In some examples, a continuous signal is collected for a predetermined period of time in response to a detected change in waveform. For example, the continuous impedance and acoustic signals may be collected for approximately 5 minutes. Signal analyzer 80 analyzes the impedance waveform (128) and analyzes the acoustic waveform (130). In some examples the analysis may be used to confirm the change in waveform previously detected. In some examples, signal analyzer 80 may determine what type of change in respiratory function has occurred.

Signal analyzer 80 may determine a plurality of impedance parameter values, including, for example, respiratory rate, respiratory variability, area under the curve of the impedance waveform (the integral of the impedance waveform), inspiration slope (the first derivative of the impedance waveform during inspiration), expiration slope (the first derivative of the impedance waveform during expiration), and mean impedance. In some examples, the area under the curve may be determined for both inspiration and for expiration. In some examples, the acoustic waveform may be analyzed to determine, for example, if a change in low frequency or high frequency amplitude has occurred. The impedance parameter values and the characteristics of the acoustic waveform may be compared to baseline values during analysis. In some examples, the baseline values may be long-term averages of the parameters.

Based on the analysis of the impedance waveform and/or the acoustic waveform, signal analyzer 80 determines whether a change in patient status has occurred (132). A change in patient status may be the occurrence of a breathing condition such as Cheyne-stokes, CSA, OSA, or pulmonary edema. In some examples, a change in patient status may be an overall increase or decrease in a patient's respiratory function. In some examples, the change between the impedance parameter values and a baseline value must be greater than a predetermined threshold to be considered a change in patient status. In some examples, patient status is determined based on at least the inspiration slope for the impedance waveform. In some examples, the patient status is determined based, at least on Table A, above in connection with table B, below.

Clinical Conditions Table B (Lung Sounds)

| Clinical | | |
|---|---|---|
| Condition | Inspiration | Expiration |
| Obstructive | Increased | Normal to |
| Sleep Apnea | Low Frequency | Slightly Elevated |
| | Acoustic Amplitude | Low Frequency |
| | | Acoustic Amplitude |
| Pulmonary | Increased | Normal to |
| Edema | High Frequency | Slightly Elevated |
| | Acoustic Amplitude | High Frequency |
| | (Crackles) | Acoustic Amplitude |
| | | (Crackles) |

In response to a determination of a change in patient status, IMD 16 stores data (136) in memory 72. The data stored in memory 72 may include the diagnosis and the impedance waveform. In some examples, the acoustic waveform or the activation event may also be stored. IMD 16 turns off the impedance and acoustic circuits (134) in response to either no change in patient status, or after the data has been stored in memory 72. IMD 16 resumes monitoring for the occurrence of an activation event.

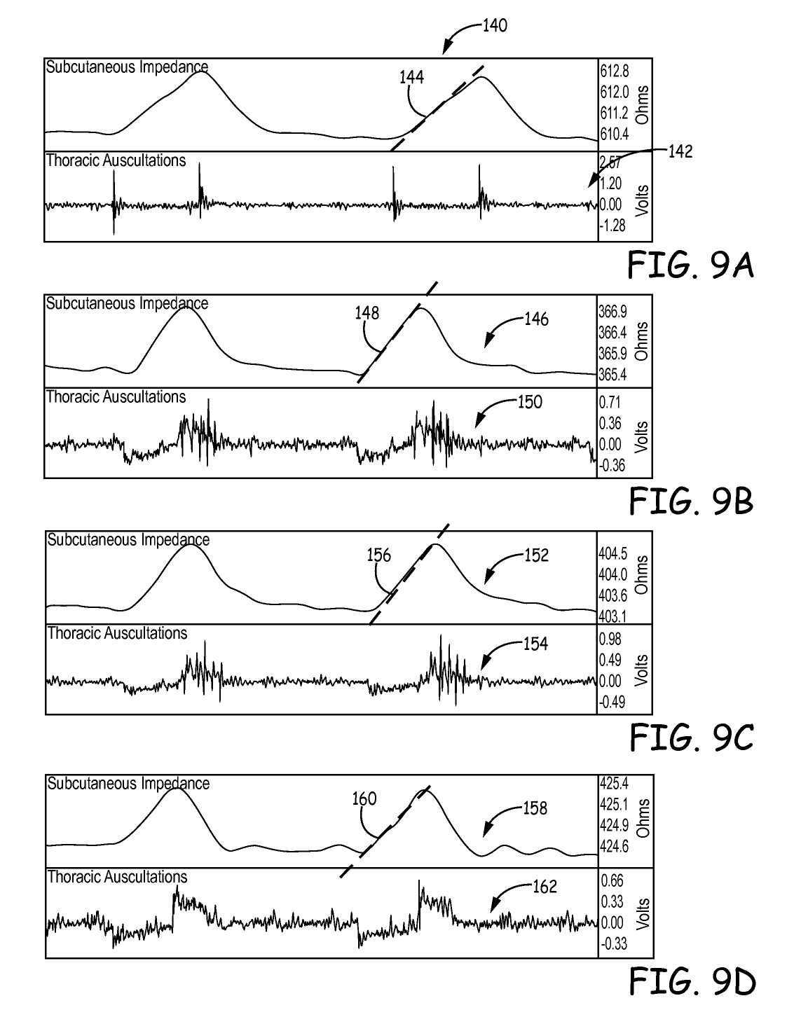

FIGS. 9A-9D are an example progression of impedance and acoustic waveforms through the diagnosis and treatment of pulmonary edema. In FIG. 9A, impedance waveform 140 is a baseline impedance waveform, and acoustic waveform 142 is a baseline acoustic waveform. Inspiration slope 144 is a baseline inspiration slope. The baseline may be collected when patient 14 is not exhibiting symptoms of pulmonary edema. In the example shown in FIG. 9A the baseline expiratory impedance is approximately 610 ohms.

In FIG. 9B, impedance waveform 146 is an impedance waveform indicative of the presence of pulmonary edema. Acoustic waveform 150 is the contemporaneous acoustic waveform. The inspiration slope 148 of the impedance waveform has increased relative to the baseline slope 144, and acoustic waveform 150 includes an increase in high frequency acoustic amplitude during inspiration relative to the baseline acoustic waveform 142. In addition, the expiratory impedance has decreased relative to baseline expiratory impedance. The expiratory impedance in FIG. 9B is approximately 365 ohms. In some examples, slope may be determined based on a detected inflection point and the change in impedance over time for a predetermined number of samples with a fixed sampling rate. A decrease in expiratory impedance may indicate passive breathing by patient 14 without active recruitment of respiratory muscles during breathing. It may also indicate a weakening in the respiratory muscles.

In FIG. 9C, impedance waveform 152 and acoustic waveform 154 illustrate patient respiratory function after one hour of the application of positive end expiratory pressure (PEEP) to treat the pulmonary edema. The inspiration slope 156 has decreased slightly relative to inspiration slope 148. The amount of high frequency noise in the acoustic waveform has also decreased after the application of PEEP. As fluid is removed from the lungs with the application of PEEP, the impedance values decrease towards baseline and the thoracic auscultations become less noisy. In addition, the expiratory impedance begins to rise towards the impedance value collected in the baseline. The expiratory impedance in FIG. 9C is approximately 403 ohms.

In FIG. 9D, impedance waveform 158 and acoustic waveform 162 shown respirator function in a patient 14 after a 1 hour recovery period without the application of PEEP. The recovery period started after PEEP had been applied for one hour. As shown in FIG. 9D, the impedance waveform continues to migrate towards the baseline values after the removal of the PEEP. As shown in FIG. 9D the expiratory impedance is 425 ohms.

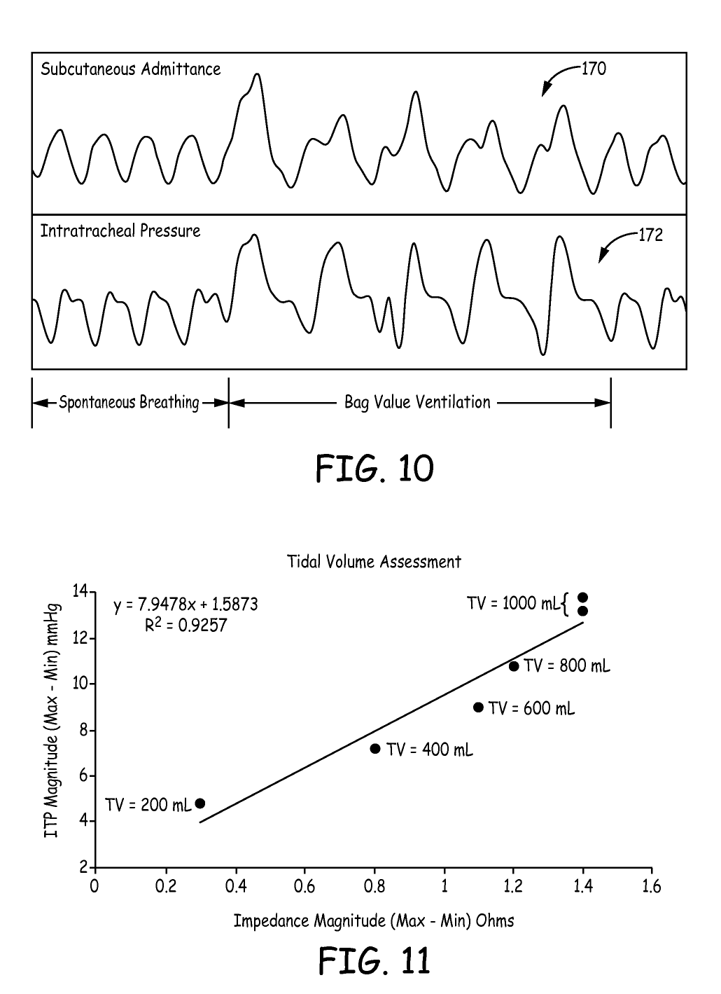

FIG. 10 is a graph of experimental data illustrating changes in real-time admittance related to intratracheal pressure. Subcutaneous admittance waveform 170 and intratracheal pressure waveform 172 were collected from a porcine over a period of time and under the same conditions. An admittance waveform is a reciprocal of an impedance waveform. That is, to determine an admittance waveform w(x) from impedance waveform z(x), w(x)=1/z(x). Thus, as used herein the term ‘impedance values” may be understood to include admittance values, i.e., values that are reciprocal of an impedance value. As illustrated in FIG. 10, the time period during which waveforms 170 and 172 were collected includes a first sub-period during which the porcine breathed spontaneously, followed by a second sub-period (5 respiratory cycles) of bag valve respiration. The bag valve respiration increased both respiratory rate and volume (thoracic expansion) relative to spontaneous breathing. As shown in FIG. 10, subcutaneous admittance 170 is sensitive to respiratory rate and change in thoracic expansion in much the same manner as intratracheal pressure. Accordingly, given the relationship between impedance and admittance, respiration parameter values may be monitored based on either an impedance or admittance waveform.

FIG. 11 is a graph illustrating experimental data relating to changes in real-time impedance related to intratracheal pressure. The graph shows the sensitivity of impedance to tidal volume change using a subcutaneous electrode array implanted in the left lateral thorax of a porcine. A positive pressure ventilator was used to adjust the tidal volume. The protocol started with a tidal volume setting at 1000 milliliters (mL) and then decreased in 200 mL increments before returning to a final setting of 1000 mL. All ventilator settings were sustained for two minutes each. As shown in FIG. 11 subcutaneous impedance as sensitive to change in tidal volume and correlates well (R²=0.93) with change in intratracheal pressure.

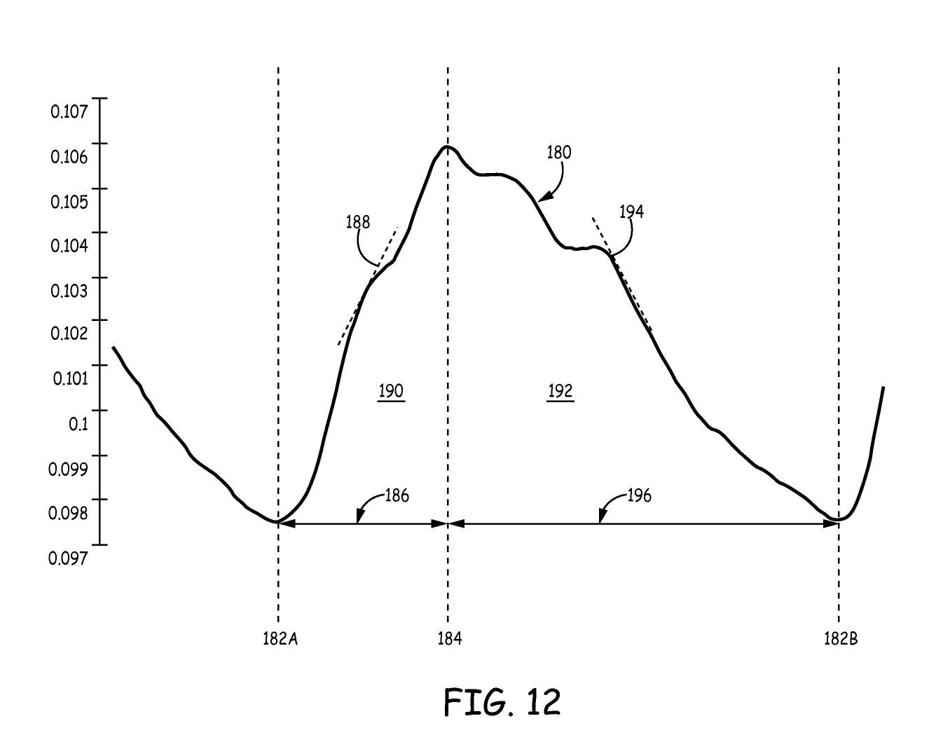

FIG. 12 is a graph illustrating an admittance waveform 180. The graph of FIG. 12 also depicts time intervals between fiducial points of admittance waveform 180. In particular, admittance waveform 180 includes fiducial points such as admittance minimum 182A, admittance maximum 184 and admittance minimum 360B for a breathing cycle. IMD 16 may detect admittance waveform 180 using any of the techniques described herein. IMD 16 may identify fiducial points such and admittance minimum 182 and admittance maximum 184. In one example, IMD 16 identifies local minima and local maxima for the current breathing cycle, e.g., between admittance minimum 182 A and admittance minimum 182B, using any known technique for identifying minima and maxima in a signal.

IMD 16 may further determine time interval 186 between admittance minimum 182A and admittance maximum 184. IMD 16 may also determine time interval 196 between admittance maximum 184 and admittance minimum 182B.

In some examples, IMD 16 may determine an average slope 188 of admittance waveform 180 during time interval 186. The average slope 188 may be the average slope during patient inspiration. IMD 16 may also determine and average slope 194 of admittance waveform 180 during time interval 196. The average slope 194 may be the average slope during patient expiration. In some examples, IMD 16 may determine an area under the curve 190 for the time interval 186. The area under the curve 190 may be the area under the curve during patient inspiration. IMD 16 may also determine an area under the curve 192 for the time interval 196. The area under the curve 192 may be the area under the curve during patient expiration. Information regarding the average inspiration slope, the average expiration slope, the area under the curve during inspiration and the area under the curve during expiration may be used to determine respiratory function, as discussed above with respect to FIGS. 7 and 8.

In some examples, dividing the respiratory waveform into an inspiration portion and an expiration portion and analyzing the area under the curve and/or slope for each portion may provide diagnostic information for changes in respiratory compliance. In addition, the ratio between the two halves may have further diagnostic value. In some examples, the ratio of the tidal volume to the inspiratory slope may be used as a respiratory compliance index. Respiratory compliance is typically the ratio of change in volume over change in pressure. In some examples, the magnitude (maximum impedance-minimum impedance) of the respiratory impedance waveform may function as a surrogate for tidal volume. The inspiratory slope (first derivative of the impedance waveform) may function as a surrogate for inspiratory pressure. Accordingly, in some examples, it may be beneficial to determine the impedance magnitude and device by the inspiratory slope first derivative. In some examples the ratio of inspiratory slope to expiratory slope may be used as an alternative respiratory compliance index.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described herein may also be embodied in a computer readable medium containing instructions. Instructions embedded in a computer readable medium may cause a programmable processor, or other processor, to perform the method, e.g. when the instructions are executed. A computer readable medium may be a computer readable storage medium. Computer readable storage media may include, for example, random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples of the present disclosure have been described. These and other examples are within the scope of the following claims.

1. A method for monitoring respiratory function, comprising:
sensing an acoustic waveform ocurring during one of an inspiration phase associated with at least one breath of a patient and an expiration phase associated with at least one breath of a patient;
determining changes in high frequency acoustic amplitude associated with the sensed acoustic waveform; and
determining an indication of respiratory function in response to the determined changes in high frequency acoustic amplitude.

2. The method of claim 1, further comprising:
determining changes in low frequency acoustic amplitude associated with the sensed acoustic waveform; and
determining the indication of respiratory function in response to the determined changes in low frequency acoustic amplitude, wherein determining an indication of respiratory function further comprises:
determining a first indication of respiratory function in response to determined changes in high frequency acoustic amplitude; and
determining a second indication of repiratory function different from the first indication of respiratory function in response to determined changes in low frequency acoustic amplitude.

3. The method of claim 2, further comprising:
receiving a first thoracic impedance waveform for at least one breath of a patient;
determining a first breath slope value in response to on the first thoracic impedance waveform; and
comparing the first breath slope value to a first threshold slope value of the patient, wherein determining an indication of respiratory function further comprises determining the indication of respiratory function in response to the comparing.

4. The method of claim 3, further comprising determining whether the first thoracic impedance waveform was collected in response to a first activation event, wherein the first threshold slope value is a first baseline slope value and wherein the first baseline slope value is associated with the first activation event.

5. The method of claim 4, further comprising:
comparing the first thoracic impedance waveform to the first baseline impedance waveform, the first baseline impedance waveform collected in response to a baseline activation event, the baseline activation event corresponding to the first activation event;
in response to the comparing the first thoracic impedance waveform to the first baseline impedance waveform, detecting a change in patient status;
in response to a detected change in patient status, associating the first thoracic impedance waveform with the new patient status; and
storing the new patient status and the first impedance waveform.

6. The method of claim 4, further comprising:
receiving a second thoracic impedance waveform;
determining whether the second thoracic impedance waveform was collected in response to a second activation event different from the first activation event;
determining a second breath slope value in response to the second thoracic impedance waveform;
comparing the second breath slope to a second baseline slope value associated with the second activation event;
comparing a difference between the first breath slope value and the first baseline slope value and a difference between the second breath slope value and the second baseline slope value; and
in response to the comparison of the differences, determining whether a change in relative respiratory function has occurred.

7. The method of claim 1, further comprising:
determining an area under the curve of the at least one breath, and
comparing the area under the curve of the at least one breath to a threshold area under the curve value; wherein determining an indication of respiratory function of the patient further comprises determining the indication of respiratory function of the patient in response to the comparison of the area under the curve of the at least one breath.

8. The method of claim 7, wherein the area under the curve for the breath is the area under the curve during inspiration.

9. The method of claim 7, wherein the area under the curve for the breath is the area under the curve for expiration.

10. The method of claim 3, wherein the first breath slope value is a long term average of inspiration slope value, the method further comprising determining a ratio between the long term average of inspiration slope value and a long term average of expiration slope value.

11. A system for monitoring respiratory function, the system comprising:
an acoustic sensing device sensing an acoustic waveform ocurring during one of an inspiration phase associated with at least one breath of a patient and an expiration phase associated with at least one breath of a patient; and
a processor configured to determine changes in high frequency acoustic amplitude associated with the sensed acoustic waveform, and determine an indication of respiratory function in response to the determined changes in high frequency acoustic amplitude.

12. The system of claim 11, wherein the processor is further configured to determine changes in low frequency acoustic amplitude associated with the sensed acoustic waveform, and determine the indication of respiratory function in response to the determined
changes in low frequency acoustic amplitude, wherein determining an indication of respiratory function further comprises:
determining a first indication of respiratory function in response to determined changes in high frequency acoustic amplitude; and
determining a second indication of repiratory function different from the first indication of respiratory function in response to determined changes in low frequency acoustic amplitude.

13. The system of claim 12, the system comprising:
an impedance sensor, the impedance sensor configured to collect a first thoracic impedance waveform for at least one breath of a patient; and
a memory configured to store a first threshold slope value, wherein the processor is further configured to determine a first breath slope value in response to the impedance waveform, and compare the first breath slope value to a first threshold slope value, wherein determining an indication of respiratory function further comprises determining the indication of respiratory function in response to the comparing.

14. The system of claim 13, wherein the processor is further configured to determine whether the first thoracic impedance waveform was collected in response to a first activation event, wherein the first threshold slope value is a first baseline slope value and wherein the first baseline slope value is associated with the first activation event.

15. The system of claim 14, wherein the processor is further configured to:
compare the first thoracic impedance waveform to the first baseline impedance waveform, the first baseline impedance waveform collected in response to an original activation event, the original activation event corresponding to the first activation event;
detect a change in patient status in response to the comparing the first thoracic impedance waveform to the first baseline impedance waveform;
in response to a change in patient status, associate the first thoracic impedance waveform with the new patient status; and
store the new patient status and the first impedance waveform in the memory.

16. The system of claim 14, wherein
the impedance sensor is further configured to collect a second thoracic impedance waveform; and
the processor further configured to:
determine whether the second thoracic impedance waveform was collected in response to a second activation event different from the first activation event;
determine a second breath slope value in response to on the second thoracic impedance waveform;
compare the second breath slope to a second baseline slope value associated
with the second activation event;
compare a difference between the first breath slope value and the first baseline
slope value and a difference between the second breath slope value and the second baseline slope value; and
in response to the comparison of the differences, determine whether a change
in relative respiratory function has occurred.

17. The system of claim 13, wherein the processor is further configured to:
determine an area under the curve of the at least one breath;
compare the area under the curve of the at least one breath to a threshold area under the curve value; and
determine the indication of respiratory function in response to the comparison of the area under the curve of the at least one breath.

18. The system of claim 17, wherein the processor is configured to determine the area under the curve during inspiration.

19. The system of claim 17, wherein the processor is configured to determine the area under the curve during expiration.

20. The system of claim 13, wherein the processor is further configured to determine a long term average of inspiration slope value, determine a long term average of expiration slope value, and determine a ratio between the long term average of inspiration slope value and the long term average of expiration slope value.

21. A computer-readable medium comprising instructions for causing a programmable processor to:
sense an acoustic waveform ocurring during one of an inspiration phase associated with at least one breath of a patient and an expiration phase associated with at least one breath of a patient;
determine changes in high frequency acoustic amplitude associated with the sensed acoustic waveform; and
determine an indication of respiratory function in response to the determined changes in high frequency acoustic amplitude.
